(19) United States
(12) Patent Application Publication
(10) Pub. No.: US 2018/0214636 A1
(43) Pub. Date: Aug. 2, 2018

(54) Smart Cartridge System For Containing And Releasing Medicament With Pumping Mechanism And Compressible Reservoir

(71) Applicant: Picolife Technologies, LLC, Jacksonville, FL (US)

(72) Inventor: Farid Amirouche, Highland Park, IL (US)

(21) Appl. No.: 15/655,873

(22) Filed: Jul. 20, 2017

Related U.S. Application Data
(63) Continuation-in-part of application No. 15/423,573, filed on Feb. 2, 2017.

(51) Int. Cl. A61M 5/172; A61M 5/14; A61M 5/145

(52) U.S. Cl. CPC A61M 5/172; A61M 5/1407; A61M 5/1723; A61M 5/14586; A61M 2205/50; A61M 2205/3334; G06F 19/3468; A61M 2230/201; A61M 2205/3368; A61M 2205/3306; A61M 2205/505; A61M 2205/52; A61M 2205/3584; A61M 2205/3569

(57) ABSTRACT

The present disclosure relates to the field of treatment of symptoms or disorders through use of a cartridge system that may be used in conjunction with a delivery system, such as an infusion set. More specifically, the present disclosure describes a communication system for delivering medicament to a user, wherein the communication system may comprise a smart cartridge, a master control unit, and a drug delivery base. A smart cartridge may comprise one or more compressible reservoirs that may contain the medicament, wherein the release of medicament from a compressible reservoir from a pumping mechanism within the cartridge. In some aspects, a smart cartridge may be controlled by a master control unit that may transmit and receive delivery data from one or more components of the smart cartridge, wherein the pumping mechanism may individually actuate a compressible reservoir and deliver medicaments continuously or at a programmable intermittent rate.

### CROSS-REFERENCE TO RELATED APPLICATION

This application is a continuation in part of and claims priority to and the full benefit of U.S. Non-Provisional patent application Ser. No. 15/423,573, filed Feb. 2, 2017, and titled “SMART CARTRIDGE SYSTEM FOR CONTAINING AND RELEASING MEDICAMENT WITH PUMPING MECHANISM AND COMPRESSIBLE RESERVOIR”, the entire contents of which are incorporated herein by reference.

### BACKGROUND OF THE DISCLOSURE

Diabetes is a complex disease caused by the body's failure to produce adequate insulin or the cell's failure to respond to insulin, resulting in high levels of glucose in the blood. Type I diabetes is a form of Diabetes Mellitus that results from autoimmune destruction of insulin-producing beta cells of the pancreas in genetically predisposed individuals. There is no current cure and treatment by injection or infusion of insulin must be continued indefinitely. Type II diabetes is a metabolic disorder brought on at any age and time by a combination of lifestyle, diet, obesity, and genetic factors. The World Health Organization recently revised its findings from a study conducted in 2004 with predictions that by 2030, 10% of the world's population of all ages will have either Type I or Type II diabetes. This translates to roughly 552 million people worldwide suffering from some form of this disease.

Typically, treatment for diabetes requires both repeated checking of blood glucose levels and several injections of insulin as prescribed by a physician throughout the day since insulin cannot be taken orally. Major drawbacks of such treatment are the constant need to draw blood and test glucose levels throughout the day, administering improper or low dosage amounts of insulin, contamination of the insulin delivery system, lifestyle or financial restrictions, the unfortunate potential development of subcutaneous scar tissue due to repeated injections at the same location, and the high cost of medication, testing strips, and other treatment related materials.

Diabetes is usually controlled by insulin replacement therapy in which insulin is delivered to the diabetic person by injection to counteract elevated blood glucose levels. Recent therapies include the basal/bolus method of treatment in which the basal, a long acting insulin medication, such as, for example, Humalog® and Apidra®, is delivered via injection once every day. The basal provides the body with an insulin profile that is relatively constant throughout the day, or could follow a profile best suited for the diabetic person. These rates can change based on the patient's response to insulin. At meal-time, an additional dose of insulin, or bolus, is administered based on the amount of carbohydrates and protein in the meal. The bolus is viewed as an emergency response to spikes in blood sugar that need to be brought down by injection of insulin. Accurate calculations of various parameters, including the amount of carbohydrates and proteins consumed and the lapse in time since the last dosage are necessary to determine the appropriate dosage of insulin. As a result, the dosages are prone to human error and the method is ineffective when doses are skipped, forgotten, or miscalculated. Exercise, stress, and other factors can also cause the calculations to be inaccurate. Bolus is usually administered when the patient's glucose level is high or above certain acceptable thresholds and requires immediate attention.

To address these problems, insulin delivery devices or pumps were developed to attempt to mimic the way a normal, healthy pancreas delivers insulin to the body. Innovations are rapidly advancing toward the creation of a closed-loop insulin delivery system. These systems employ real-time glucose-responsive insulin administration via continuous glucose monitoring and wireless communication with a controller which dispenses insulin based on tightly controlled algorithms. The two main algorithmic systems used to calculate insulin dosages automatically are the proportional-integral-derivative (PID) control, or and the mathematic-predictive control (MPC). MPC algorithms can be considered proactive or predictive. They forecast glucose levels in anticipation of meals, physical activity and administer insulin over a prediction window of 1.5 to 3 hours or longer. PID algorithms are considered reactive in response to measured glucose levels and cannot predict dosages. Unfortunately, there is currently no industry-wide standard in place for embedded algorithmic calculations, and dose calculations vary from device to device.

Often, both methods are utilized when insulin is co-administered with glucagon or other medication, though silico simulations, or computer simulated, glycemic regulation via MPC calculations achieves superior glucose regulation.

Most insulin pumps today are programmed to deliver a continual basal dose of insulin and occasionally a bolus dose, usually performed manually, in response to a patient's meal intake and physical activities. Early pumps had many limitations which made them inconvenient and less effective. Their overall size, propensity to leak, and extremely high cost made them unusable for long-term disease management and financially out of reach for most patients with limited or no insurance coverage. These types of pumps were also potentially risky in terms of unintentionally over or under dosing a patient, because the accuracy of the dose administered is dependent upon the reliability of the piston-driven motor, and medication is delivered in quick bursts rather than diffused over time.

Conventional insulin pumps are worn outside the body and are connected to the user via a cannula that is inserted somewhere on the user's abdomen. The insulin is delivered under the skin and is absorbed into the body through the subcutaneous fat layer. Subcutaneous delivery of insulin takes advantage of the lack of blood flow in this area which allows for slower absorption of the medication through the dermal capillaries. Other methods of non-invasive insulin delivery have been explored and compared in Various Non-Injectable Delivery Systems for the Treatment of Diabetes Mellitus, Yadav, N., Morris, G., Harding, S., Ang, S., Adams, G., Endocrine, Metabolic & Immune Disorders-Drug Targets, 2009, Vol. 9 (1):1-13. The pump is worn on the user's body at all times, concealed by clothing as desired, and therefore should be as small and unobtrusive as possible. The tubing connecting the pump to the user must be relatively short as crystallization of the insulin medication is of great concern when the tubing is long.

One recurring problem with most miniaturized ambulatory infusion pumps available today is that the amount of medication which can be stored in the reservoirs often cannot meet the needs of certain diabetic patients. Many Type II diabetics who require insulin often need more insulin per gram of carbohydrate due to a condition referred to as “insulin resistance.” Additionally, many diabetic therapies include one or more medications delivered alternately or simultaneously. For this reason, a medication pump which employs a plurality of reservoirs able to dispense medication at variable rates is optimal. Therefore, a substantial need exists to best maximize the volume of the medication reservoirs while maintaining a very small overall size of the device itself.

With the demand for a decrease in size of the pump unit also comes a decreased size in the medication reservoir. This reduced reservoir size means more frequent refilling, greater potential for contamination of the reservoir, more frequent changes of the cannula and tubing, and greater expense overall in treating the condition. Frequent manual refilling of a medication reservoir can also lead to the increased formation of bubbles, which is a significant problem. Even very small bubbles of 10 microliters or less can displace enough fluid to equal a missed dose of 1 unit of medicament. Insulin medication itself can also form bubbles when dissolved air is “outgassed” through normal changes in temperature or atmospheric pressure. Therefore the need exists to provide a disposable, pre-pressurized, pre-filled medication reservoir that can work as part of a medication pump system to provide extremely accurate delivery of a plurality of medications.

### SUMMARY OF THE DISCLOSURE

What is needed is a smart cartridge that addresses the concerns laid out above while delivering an insulin treatment protocol that delivers on a variety of factors. The future of the insulin treatment protocol detailed above is vitally dependent upon several factors: more accurate glucose sensors, rapid response software and hardware, catheters with multiple delivery channels for both glucose sensing and medication diffusion, and dual or multi-chambered medication delivery cartridge systems. The present invention meets these current and future needs.

The present disclosure relates to the field of treatment of symptoms or disorders through use of a multifunctional cartridge system that may be used in conjunction with a drug delivery base, such as an infusion set with multiple channels and an adjustable base for subcutaneous interface and delivery. More specifically, the present disclosure describes a smart cartridge for containing and releasing medicament, wherein the smart cartridge may comprise a system that may be operable when digital signals are produced through an electrical communication with a power source within a master control unit.

In some aspects, the present disclosure relates to a drug delivery communication system for use in conjunction with a smart cartridge system, wherein the communication system may comprise a master control unit device comprising a first electronic receiving body, a power source, a master control processor, wherein the master control processor is configured to control the power source; a first smart cartridge device connectable to the master control unit, wherein communication initiates when the first smart cartridge is inserted into the first electronic receiving body, the first smart cartridge device comprising a first compressible reservoir configured to contain a first medicament; a first internal tubing connected to the first compressible reservoir; a pumping mechanism operably connected to the first internal tubing and configured to be electrically connected to the power source, wherein the pumping mechanism controls flow of the first medicament from the first compressible reservoir; a flow rate sensor configured to monitor a flow of the first medicament from the first internal tubing to the first outlet tube; a first outlet tube connected to the first internal tubing through which a first expulsion of the first medicament to an external body flows; and a housing containing the first compressible reservoir, the first internal tubing, the pumping mechanism, the flow rate sensor, and at least part of the first outlet tube; and a first smart cartridge processor configured to receive a first set of delivery data from the master control processor and transmit a second set of delivery data to the master control processor, wherein the first set and the second set of delivery data at least partially controls the delivery of the first medicament to a user and the delivery of power to the first smart cartridge.

In some embodiments, at least a portion of the drug delivery device communication is wireless. In some aspects, the second set of delivery data may comprise data from one or more the first compressible reservoir, the first internal tubing, the pumping mechanism, the flow rate sensor, the first outlet tube, the housing, and the first smart cartridge processor. In some implementations, the pumping mechanism may comprise a pump actuation system with a pump processor configured to receive pump data from one or both the master control processor and the first cartridge processor, wherein the pump data controls an actuation of the pump actuation system.

In some aspects, the second set of delivery data may comprise delivery data from one or both the pump actuation system and pump processor. In some implementations, the master control unit may be configured to receive a first set of status data from the first smart cartridge device, wherein the first set of status data may relate to a status of one or more the master control unit, the first compressible reservoir, the first internal tubing, the pumping mechanism, the flow rate sensor, the first outlet tube, the housing, and the first smart cartridge processor.

In some embodiments, the drug delivery communication system may further comprise a drug delivery base in communication with one or both the smart cartridge and the master control unit. In some aspects, the drug delivery base may comprise a first drug delivery base processor configured to transmit a third set of delivery data from the master control processor, wherein the delivery data at least partially controls the delivery of the first medicament to a user; a first receiving channel connectable to the first outlet tube; a first dispensing channel configured to deliver medicament subcutaneously; a platform configured to secure to a skin surface of a user, wherein the platform secures the first outlet tube to the first dispensing channel and stabilizes the first dispensing channel to the user.

In some aspects, the status data may further relate to a status of one or more of the first drug delivery base processor, the first receiving channel, the first dispensing channel, and the platform, and wherein the second set of delivery data comprises delivery data from one or more of the first drug delivery base processor, the first receiving channel, the first dispensing channel, and the platform. In some embodiments, the first compressible reservoir may comprise a first fill port configured to accept a filling mechanism, wherein the filling mechanism is configured to add a first medicament to the first compressible reservoir; a first overflow port configured to dispel an excess amount of the first medicament where the first medicament filled exceeds a first threshold volume capacity within the first compressible reservoir; a first flow port through which the first medicament flows for use; and a first flexible pouch configured to contain the first medicament.

In some implementations, the status data may further relate to a status of one or more of the first fill port, first overflow port, first flow port, and the first flexible pouch, and wherein the second set of delivery data comprises delivery data from one or more of the first fill port, first overflow port, first flow port, and the first flexible pouch. In some aspects, the master control unit device may further comprise an interface configured to receive delivery input from the user, wherein the delivery data is based at least in part on the delivery input. In some embodiments, the drug delivery communication system may further comprise a first external communication device configured to receive one or more the first set of status data, the first set of delivery data, the second set of delivery data, and the third set of delivery data.

In some aspects, a drug delivery communication system for use in conjunction with a smart cartridge system may comprise a master control unit device comprising a first electronic receiving body, a power source, a master control processor, wherein the master control processor is configured to control the power source; a smart cartridge device connectable to the master control unit, wherein the smart cartridge comprises a first compressible reservoir configured to contain a first medicament; a first internal tubing connected to the first compressible reservoir; a second compressible reservoir configured to contain a second medicament; a second internal tubing connected to the second compressible reservoir; a pumping mechanism operably connected to the first internal tubing and the second internal tubing and configured to be electrically connected to the power source, wherein the pumping mechanism controls flow of the first medicament from the first compressible reservoir and flow of the second medicament from the second compressible reservoir; a first outlet tube connected to the first internal tubing through which a first expulsion of the first medicament to an external body flows; a second outlet tube connected to the second internal tubing through which a first expulsion of the second medicament to the external body flows; and a flow rate sensor configured to monitor a flow of the first medicament from the first internal tubing to the first outlet tube and a flow of the second medicament from the second internal tubing to the second outlet tube; a housing containing the first compressible reservoir, the first internal tubing, the pumping mechanism, the flow rate sensor, and at least part of the first outlet tube; a first smart cartridge processor configured to receive a first set of delivery data from the master control processor and transmit a second set of delivery data to the master control processor, wherein the first set and the second set of delivery data at least partially controls the delivery of the first medicament and the second medicament to a user and the delivery of power to the first smart cartridge.

In some embodiments, the second set of delivery data may comprise data from one or more the first compressible reservoir, the second compressible reservoir, the first internal tubing, the second internal tubing, the pumping mechanism, the flow rate sensor, the first outlet tube, the second outlet tube, the housing, and the first smart cartridge processor. In some aspects, the pumping mechanism may comprise a pump actuation system with a pump processor configured to receive pump data from one or both the master control processor and the first cartridge processor, wherein the pump data controls an actuation of the pump actuation system.

In some implementations, the master control unit may be configured to receive a first set of status data from the first smart cartridge device, wherein the first set of status data relates to a status of one or more the master control unit, the first compressible reservoir, the second compressible reservoir, the first internal tubing, the second internal tubing, the pumping mechanism, the flow rate sensor, the first outlet tube, the second outlet tube, and the housing, the first smart cartridge processor.

In some aspects, the drug delivery communication system may further comprising a drug delivery base in communication with one or both the smart cartridge and the master control unit, the drug delivery base comprising a first drug delivery base processor configured to transmit a third set of delivery data from the master control processor, wherein the delivery data at least partially controls the delivery of the first medicament to a user; a first receiving channel connectable to the first outlet tube; a first dispensing channel configured to deliver the first medicament subcutaneously; a second receiving channel connectable to the second outlet tube; a second dispensing channel configured to deliver the second medicament subcutaneously; a platform configured to secure to a skin surface of a user, wherein the platform secures the first outlet tube to the first dispensing channel, the second outlet tube to the second dispensing channel and stabilizes the first dispensing channel and the second dispensing channel to the user.

In some implementations, the status data may further relate to a status of one or more of the first drug delivery base processor, the first receiving channel, the first dispensing channel, and the platform, and wherein the second set of delivery data comprises delivery data from one or more of the first drug delivery base processor, the first receiving channel, the first dispensing channel, and the platform. In some aspects, the master control unit device may further comprise an interface configured to receive delivery input from the user, wherein the delivery data is based at least in part on the delivery input. In some aspects, the drug delivery communication system may further comprise a first external communication device configured to receive one or more the first set of status data, the first set of delivery data, the second set of delivery data, and the third set of delivery data.

In the following sections, detailed descriptions of examples and methods of the disclosure will be given. The description of both preferred and alternative examples though thorough are exemplary only, and it is understood that to those skilled in the art variations, modifications, and alterations may be apparent. It is therefore to be understood that the examples do not limit the broadness of the aspects of the underlying disclosure as defined by the claims.

### BRIEF DESCRIPTION OF THE DRAWINGS

The accompanying drawings that are incorporated in and constitute a part of this specification illustrate several embodiments of the disclosure and, together with the description, serve to explain the principles of the disclosure:

FIG. 1A illustrates a perspective view of an exemplary smart cartridge with two compressible reservoirs and a single outlet, in accordance with some embodiments of the present disclosure.

FIG. 1B illustrates a perspective view of an exemplary smart cartridge with two compressible reservoirs and a single outlet, in accordance with some embodiments of the present disclosure.

FIG. 1C illustrates a perspective view of an exemplary smart cartridge with two compressible reservoirs and a single outlet, in accordance with some embodiments of the present disclosure.

FIG. 2 illustrates a perspective view of an alternate exemplary smart cartridge with two compressible reservoirs and two Luer-Lock outlets, in accordance with some embodiments of the present disclosure.

FIG. 3 illustrates a perspective view of an alternate exemplary embodiment of a smart cartridge with upper housing and lower housing, wherein the smart cartridge comprises reservoirs with different volume capacities, in accordance with some embodiments of the present disclosure.

FIG. 4 illustrates a perspective view of an exemplary smart cartridge with a single compressible reservoir, in accordance with some embodiments of the present disclosure.

FIG. 5A illustrates an alternate exemplary embodiment of a smart cartridge with a single compressible reservoir, wherein the single compressible reservoir comprises a dual port end cap, in accordance with some embodiments of the present disclosure.

FIG. 5B illustrates an alternate exemplary embodiment of a smart cartridge with a single compressible reservoir, wherein the single compressible reservoir comprises a dual port end cap, in accordance with some embodiments of the present disclosure.

FIG. 6A illustrates an exemplary embodiment of a pumping mechanism engaged with an external control unit, wherein control unit electromagnets may control the stroke of pump magnets interacting through a membrane, in accordance with some embodiments of the present disclosure.

FIG. 6B illustrates an exemplary embodiment of a pumping mechanism engaged with an external control unit, wherein control unit electromagnets may control the stroke of pump magnets interacting through a membrane, in accordance with some embodiments of the present disclosure.

FIG. 7A illustrates an exemplary embodiment of a flow rate sensor, wherein the flow rate sensor may detect flow of a medicament from a compressible reservoir through an outlet tube, in accordance with some embodiments of the present disclosure.

FIG. 7B illustrates an exemplary embodiment of a flow rate sensor, wherein the flow rate sensor may detect flow of a medicament from a compressible reservoir through an outlet tube, in accordance with some embodiments of the present disclosure.

FIG. 8 illustrates exemplary embodiments of wireless circuit boards of a master control unit and a smart cartridge, wherein the smart cartridge may be inserted into the master control unit allowing for electrical communication between the smart cartridge and the master control unit, in accordance with some embodiments of the present disclosure.

FIG. 9 illustrates an exemplary drug delivery communication system.

FIG. 10A illustrates a side view of an exemplary drug delivery communication system.

FIG. 10B illustrates a side view of an exemplary drug delivery communication system.

FIG. 11A illustrates a perspective view of a drug delivery communication system.

FIG. 11B illustrates a perspective view of a drug delivery communication system.

FIG. 11C illustrates a perspective view of a drug delivery communication system.

FIG. 12 illustrates a perspective view of an exemplary drug delivery base.

FIG. 13 illustrates exemplary communication exchange within a drug delivery communication system and with external devices.

FIG. 14 illustrates an exemplary cyber physical healthcare system.

FIG. 15 illustrates an exemplary block diagram of an exemplary embodiment of a mobile device.

### DETAILED DESCRIPTION

The present disclosure relates to the field of a smart cartridge with dual reservoirs with an integrated pump actuation mechanism and collapsible capacitance controlled reservoirs. The smart cartridge includes a disposable pump with refill options on independent ports for delivery and control of more than one medicament such as insulin, glucagon, or a combination of therapeutic agents for the treatment management of type 1 and type 2 diabetic patients. More particularly, the disclosure relates to dual pump sensory cartridge pump devices with a microcontroller, feedback control, and self-monitoring of fluidic delivery. The current invention relates to the cartridge system with active control valves, along with volumetric flow sensors integrated into a dual chamber pump for storing and delivering medicament or other therapeutic agents for the treatment and management of ailments, such as, for example, diabetes or chronic pain.

The present disclosure relates to improving the use of medicament pumps to transport medicaments from a compressible reservoir to a patient such as through an infusion set, for delivery of insulin or other medicaments to a patient. More particularly, the disclosure relates to a smart cartridge of a medicament pump where the medicament reservoir and pump mechanism are combined into a single, cost-effective unit. In some embodiments, the pump cartridge unit may be a single-use disposable component configured to interact with a reusable pump or medicament distribution system. In some exemplary embodiments, the pump cartridge unit may be configured to prevent repeated uses, thereby ensuring the pump cartridge is disposable. In other embodiments, reservoirs with the pump cartridge unit may be refillable.

In the following sections, detailed descriptions of examples and methods of the disclosure will be given. The description of both preferred and alternative examples, though thorough, are exemplary only, and it is understood to those skilled in the art that variations, modifications, and alterations may be apparent. It is therefore to be understood that the examples do not limit the broadness of the aspects of the underlying disclosure as defined by the claims.

### Glossary

Master Control Unit: as used herein refers to a master control device that may communicate and control components within a drug delivery communication system. In some aspects, a master control unit may receive a smart cartridge, wherein the master control unit may provide adjustable power to the pump actuation system in the smart cartridge. In some embodiments, a master control unit may coordinate the transfer of medicament from the smart cartridge through a drug delivery base and into the body of a user.
Pump Actuation System: as used herein refers to a pumping system, which may be magnetically driven, that may direct fluid through actuation of one or more valve membranes. In some aspects, a pump actuation system may be integrated with a smart cartridge, wherein the pump actuation system may be controlled and operated by a master control unit.
Drug Delivery Base: as used herein refers to a device that may direct medicament received from one or both a master control unit and smart cartridge into the body of a user. In some aspects, a drug delivery base may comprise a platform that may secure and house at least a portion of the directing mechanisms, such as cannulae or catheter with multiple delivery channels. In some embodiments, the platform may be adhered to the skin of a user, which may decrease shifting of the directing mechanisms through use of the drug delivery base. In some embodiments, a drug delivery base may comprise additional functionality, such as corrective engine points, sensors, or secondary control, as non-limiting examples.
Smart Cartridge: as used herein refers to a system for containing and releasing medicament, wherein the system may be operable when in electrical communication with an external power source and master control unit. In some embodiments, a cartridge may comprise one or more compressible tailored reservoirs that may contain the medicament, wherein the release of medicament from a compressible reservoir with a T connector check valve operation and may be controlled by a pumping mechanism within the cartridge. In some aspects, a cartridge may be operable when paired or connected with a master control unit or other device or system comprising a power source and control mechanism, such as a graphical user interface (sometimes referred to as a “GUI”) through a portable communication device, wherein the pumping mechanism may individually actuate a compressible reservoir and deliver medicaments continuously at a controlled speed or at a programmable intermittent rate. As used herein, the term smart cartridge may be contrasted with a common use of the term cartridge traditionally used in medicament pumps, which typically comprises a passive container of the medicament.
Compressible Reservoir: as used herein refers to a partially flexible reservoir contained within the smart cartridge that may compress to the volume of medicament contained through controlled channels, connectors, and valves, wherein drawing medicament may compact adjacent walls of the flexible portion of the compressible reservoir and adding medicament may enlarge the interior of the flexible portion of the compressible reservoir. As used herein, a compressible reservoir may be contrasted with a traditional cartridge, which is typically rigid, and more recent soft reservoir embodiments, which may inflate and deflate like a balloon or bladder.

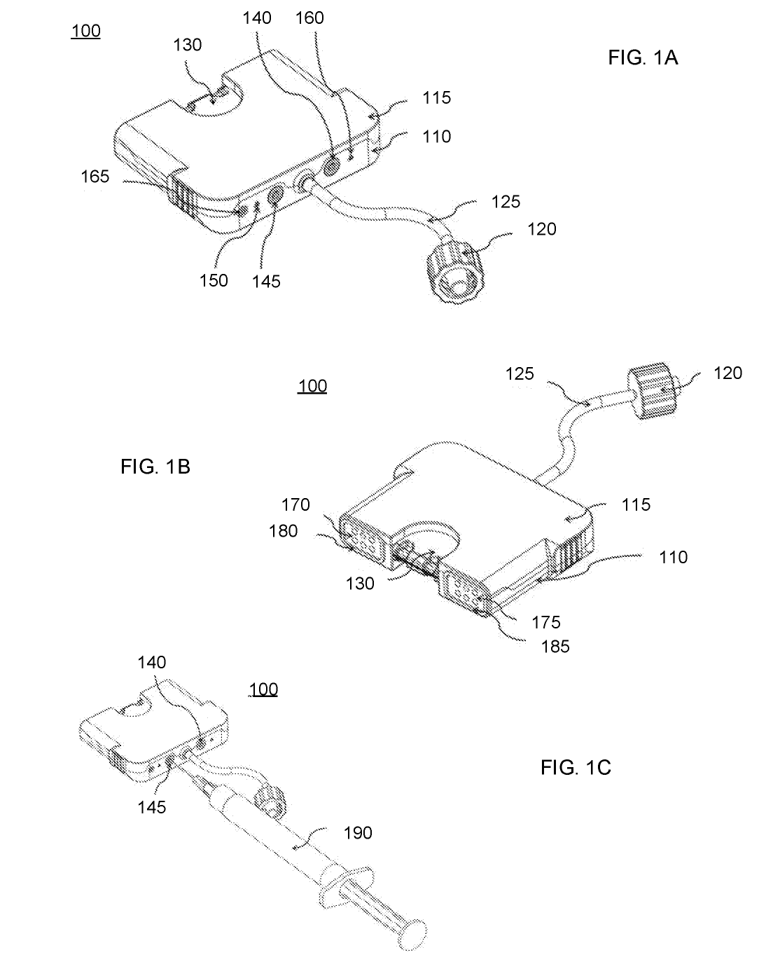

Referring now to FIGS. 1A-1C, perspective views of an exemplary smart cartridge 100 is illustrated. In some aspects, the smart cartridge 100 may comprise a first case part 110 and a second case part 115, wherein connecting, fitting, or combining the first case part 110 and the second case part 115 may create a cavity, which may house and contain one or more of the components of the smart cartridge 100. In some embodiments, the first case part 110 and the second case part 115 may comprise a substantially square or rectangular configuration. In some aspects, the first case part 110 and the second case part 115 may be permanently bonded, such as through glue, welding, magnets, slotted inserts, or other adhesive means.

In some implementations, the smart cartridge 100 may comprise a pump mechanism 130 that may release medicament through a single outlet tube 125 with a Luer-Lock male fitting 120, which may be attached to a female fitting on an infusion set (not pictured). The smart cartridge 100 may further comprise a plurality of target connectors 170, 175 with a plurality of sealing gaskets 180, 185, as seen in FIG. 1B, which may secure placement within a pump display unit (not shown). The target connectors 170, 175 and sealing gaskets 180, 185 may ensure alignment and connection with electrical connectors and spring-loaded pins of a pump display unit, such as illustrated in FIG. 20.

In some aspects, the smart cartridge 100 may comprise refill ports 140, 145, wherein a syringe 190 may be inserted through the refill ports 140, 145 to inject medicament into the compressible reservoirs, as seen in FIG. 1C. In some implementations, overflow holes 160, 150 may provide a safety system to reduce overpressure, particularly during a refill. One or both the first case part 110 and the second case part 115 may comprise a plurality of through holes 165, which may be covered in the inside of the cartridge by a breathing membrane configured to allow passage of substances with predefined characteristics, for example, certain fluids, such as air or other gases, into and out of the smart cartridge 100, which may facilitate deflation or collapsing of reservoirs.

In some aspects, reservoirs may be filled or refilled through refill ports 140, 145 present in the first case part 110 with the aid of any suitable device, such as a syringe 190 configured to fluidly connect to the refill ports, such as shown in FIG. 1C. The refill ports 140, 145 may accommodate a variety of syringe types of diverse needle length and diameter, and may be easily distinguishable by the user to ensure refill of the compressible reservoir units with the correct drugs. For example, there may be distinctive shapes, design or colors, and may be marked with the name or type of drug to be used).

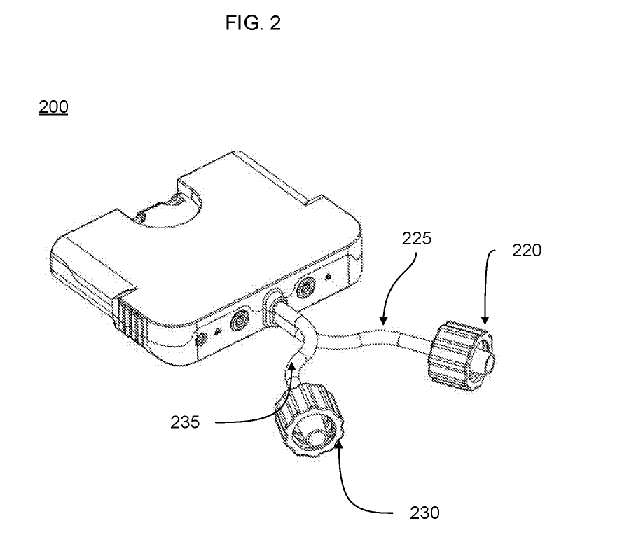

Referring now to FIG. 2, a perspective view of an alternate exemplary embodiment of a smart cartridge 200 is illustrated, wherein the smart cartridge 200 comprises two outlet tubes 225, 235, each with a separate Luer-Lock fitting 220, 230, respectively. In some aspects, the two outlet tubes 225, 235 may extend from the smart cartridge in an overlapping arrangement to limit tangling of the outlet tubes 225, 235. In some embodiments (not shown), the two outlet tubes 225, 235 may extend from different locations on the smart cartridge 200, wherein the different locations may allow for easy attachment of the Luer-Lock fittings 220, 230 to infusion sets that may be worn on separate locations of the body, such as a leg and a torso.

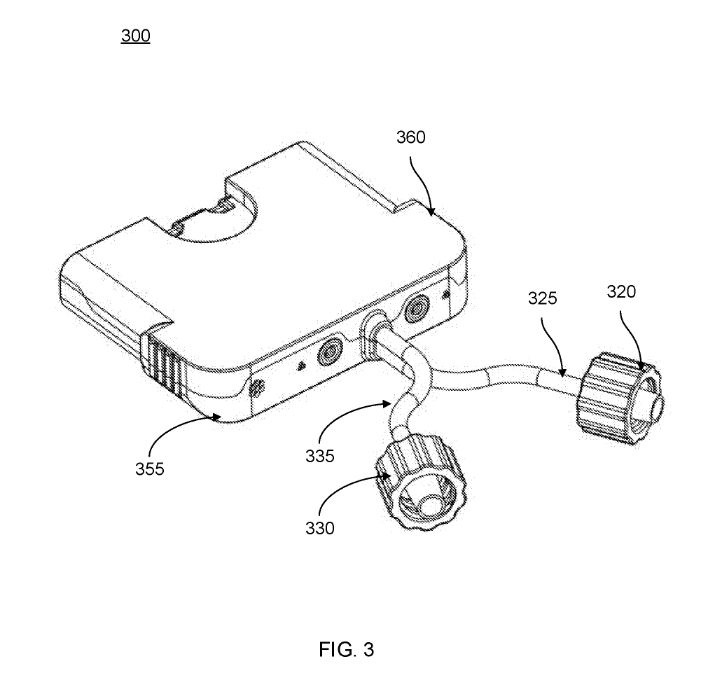

Referring now to FIG. 3, a perspective view of an alternate exemplary embodiment of a smart cartridge 300 with upper housing 360 and lower housing 355 is illustrated, wherein the smart cartridge 300 comprises reservoirs with different volume capacities. In some aspects, the smart cartridge 300 may comprise two outlet tubes 325, 335, each with a separate Luer-Lock fitting 320, 330, respectively, wherein each of the outlet tubes 325, 335 may extend from separate reservoirs within the smart cartridge 300. Where the compressible reservoirs may comprise different shapes and/or different volume capacities, the housing 355, 360 may be asymmetrical, wherein the outlet tubes 325, 335 may be held off-center within the smart cartridge 300.

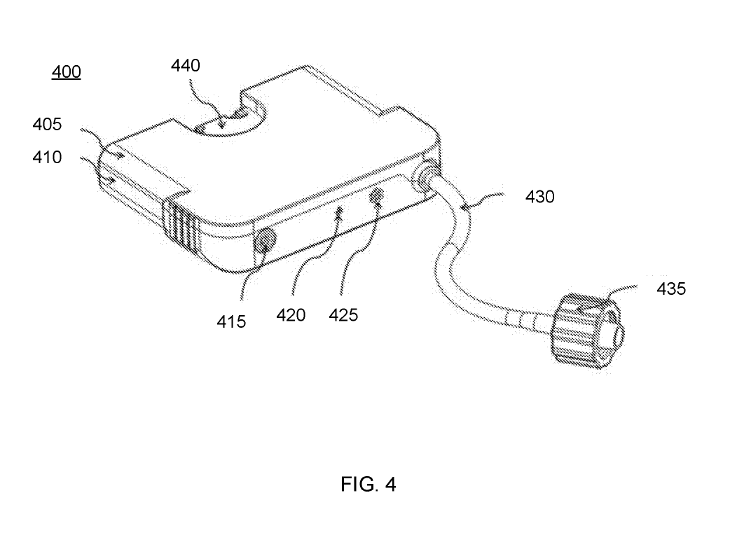

Referring now to FIG. 4, a perspective view of an exemplary smart cartridge 400 with a single compressible reservoir is illustrated. In some aspects, a smart cartridge 400 with a a pumping mechanism 440 and a single compressible reservoir may comprise similar features to a smart cartridge with dual compressible reservoirs, such as illustrated in FIGS. 1A-1C. In some embodiments, the smart cartridge 400 may comprise a fill port 415 and an overflow port 420. The components of the smart cartridge 400 may be contained within an upper housing 405 and a lower housing 410. In some aspects, one or both the upper housing 405 and the lower housing 410 may comprise an opening for an outlet tube 430 with a male Luer-Lock fitting 435 and through holes 425, which may reduce collection of moisture or build-up of gas pressure.

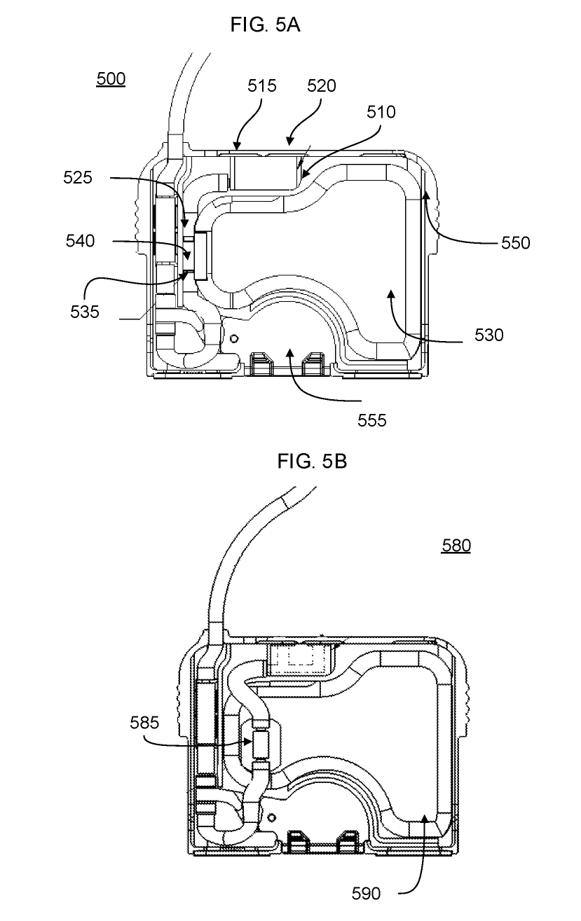

Referring now to FIG. 5A, an alternate exemplary embodiment of a smart cartridge 500 with a single compressible reservoir 530 is illustrated, wherein the single compressible reservoir 530 comprises a dual port end cap 540. In some aspects, a dual port end cap 540 may allow for a larger threshold volume capacity of the compressible reservoir 530 in a similar sized housing 550 as a compressible reservoir with two end caps. The dual port end cap 540 may comprise an intake port 525 and a flow port 535. In some aspects, a pumping mechanism 555 may draw medicament from the compressible reservoir 530 through internal tubing connected to the flow port 535. In some embodiments, the intake port 525 may be connected to a fill fitting 510, which may comprise a fill port 520 and an overflow port 515.

Referring now to FIG. 5B, an alternate exemplary embodiment of a smart cartridge 580 with a single compressible reservoir 590 is illustrated, wherein the single compressible reservoir 590 comprises a dual port fitting 585. In some implementations, the dual port fitting 585 may be located on the front of the compressible reservoir 590, which may efficiently share space within the smart cartridge 580. The efficient use of space may allow for a compressible reservoir 590 with a higher volume capacity.

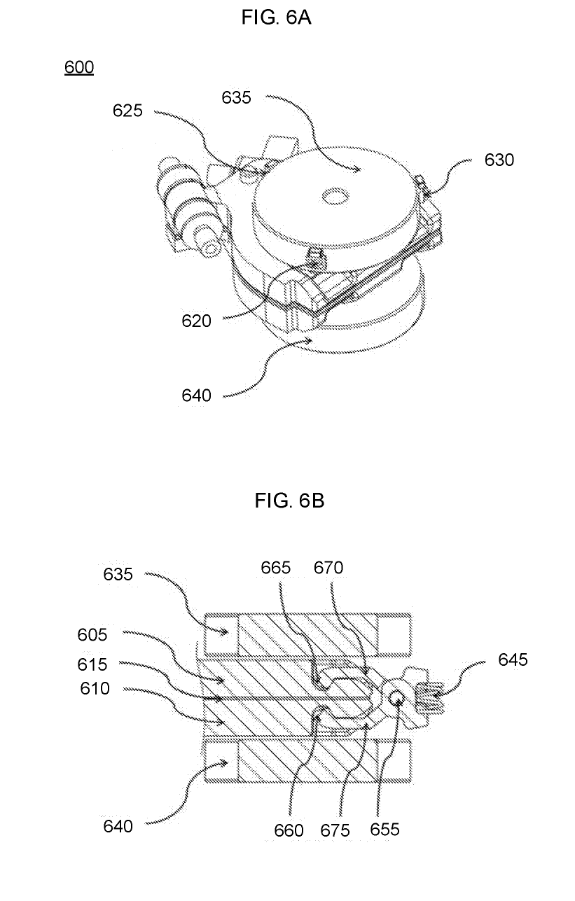

Referring now to FIGS. 6A-6B, an exemplary embodiment of a pumping mechanism 600 engaged with an external control unit is illustrated, wherein control unit electromagnets 635, 640 may control a stroke of pump magnets 605, 610 interacting through a membrane 615. In some aspects, the external control unit may comprise a locking mechanism 655 with a spring 645 and clamping 670, 675 that may engage with cartridge grooves 660, 665. In some embodiments, the control unit may comprise a plurality of triaxial Hall effect sensors 620-630.

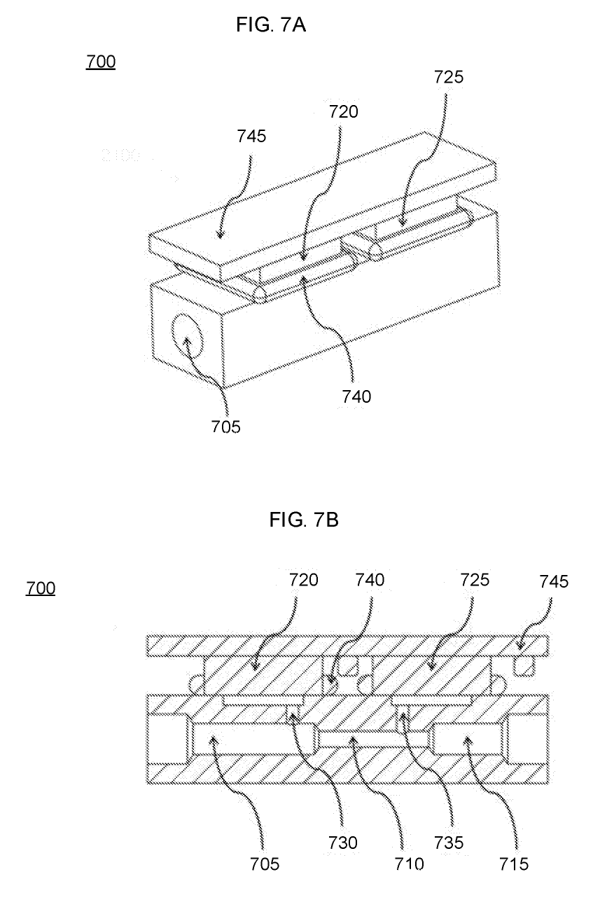

Referring now to FIGS. 7A-7B, an exemplary embodiment of a flow rate sensor 700 is illustrated, wherein the flow rate sensor 700 may detect flow of a medicament from a compressible reservoir through an outlet tube. In some aspects, the flow rate sensor 700 may measure and calculate a dual flow rate. In some embodiments, a fluid at original pressure may be pushed through a first channel 705, through a second channel 710 reaching a second pressure, and finally through a third channel 715. In some aspects, the second channel 710 may have a diameter less than the first channel 705. For example, the first channel 705 may have, by way of example, a diameter of 0.031 inch and the second channel 710 may have, by way of example, a diameter of 0.015 inch.

In some implementations, the flow rate sensor 700 may comprise one or more pressure sensors 720, 725, wherein the pressure sensors 720, 725 may measure the drop in pressure between the first channel 705 and the second channel 710. The Venturi effect may allow for the calculation of the volumetric flow rate based on the first measured pressure by the first fluid pressure sensor 720 through a first measurement channel 730 and the second measured pressure by the second fluid pressure sensor 725 through a second measurement channel 735.

In some aspects, pressure sensors 720, 725 may be hermetically bonded, such as through use of bonded joints 740, to the body of the flow rate sensor 700. In some embodiments, the measurement channels 730, 735 may be filled with a biocompatible gel that may insulate the pressure sensors 720, 725 from the fluid, wherein the insulation may increase the sensitivity of the flow rate sensor 700, which may enhance its accuracy. In some aspects, a printed circuit board 745 may be mounted and attached onto the pressure sensors 725, 720, which may allow for communication between the flow rate sensor 700 and a microcontroller unit.

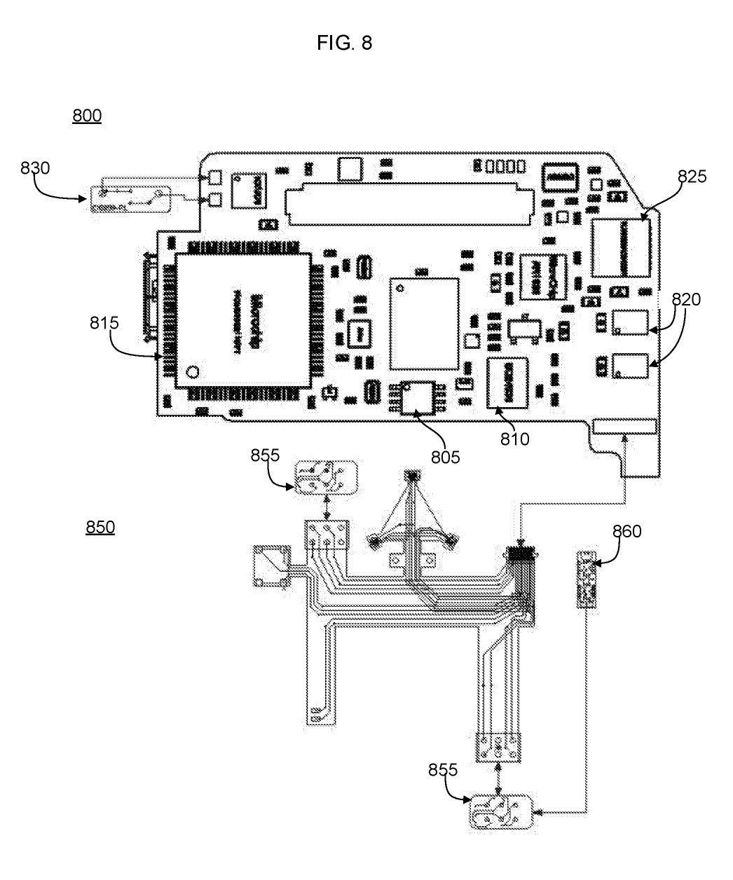

Referring now to FIG. 8, exemplary embodiments of wireless circuit boards of a pump display unit (PDU) 800 and a smart cartridge 850 are illustrated, wherein the smart cartridge 850 may be inserted into the PDU 800 allowing for electrical communication between the smart cartridge 850 and the PDU 800. In some embodiments, the PDU 800 may comprise a battery printed circuit board (“PCB”) 830, which may control the power of one or both the PDU 800 or the smart cartridge 850. In some aspects, the PDU 800 may comprise a microcontroller 815, an audio digital to analog converter 825, real time clock 805, and a charge control 810. In some embodiments, the PDU 800 may comprise one or more pump drivers 820, wherein the pump drivers 820 may control at least some of the functionality of the pump mechanism on the smart cartridge 850.

In some aspects, the smart cartridge 850 may comprise a flow sensor PCB 860, which may interface with the flow rate sensor. The smart cartridge 850 may comprise one or more cartridge PCBs 855, which may control and process data from the smart cartridge 850. In some implementations, each cartridge PCB 855 may manage and control individual reservoirs.

In some aspects, the PDU 800 and the smart cartridge 850 may share the processing and control of the components of one or both the PDU 800 and the smart cartridge 850. For example, a battery PCB 830 of the PDU 800 may provide power to the smart cartridge 850, allowing the flow sensor PCB 860 to process the flow rate sensor data.

In some embodiments, the smart cartridge 850 may process a number of calculations and then share the processed data with the PDU 800. In some aspects, the smart cartridge 850 may comprise a sensor interface that may detect one or both the number of reservoirs and the capacities of each reservoir, which may allow for interchangeability between smart cartridges 850 with different reservoir quantities and volume capacities. Such flexibility on the smart cartridge 850 may reduce the need for different PDUs 800 as the smart cartridge 850 may transmit specifications to the PDU 800, allowing for informed control of the pumping mechanism.

In some aspects, the smart cartridge 850 may be filled and/or refilled with a variety of subcutaneous drugs. Once inserted into the PDU 800, the smart cartridge 850 may deliver medicaments such as insulin, which may include long or slow acting options to lower the patient's blood glucose level, amylin analogues such as Pramlintide, or glucagon to raise the patient's blood glucose level. As another illustrative example, the smart cartridge 850 may deliver subcutaneous medication for pain management. The subcutaneous infusion of a range of liquid medications may be possible with the combination of the PDU 800 and a smart cartridge 850 inserted into the PDU.

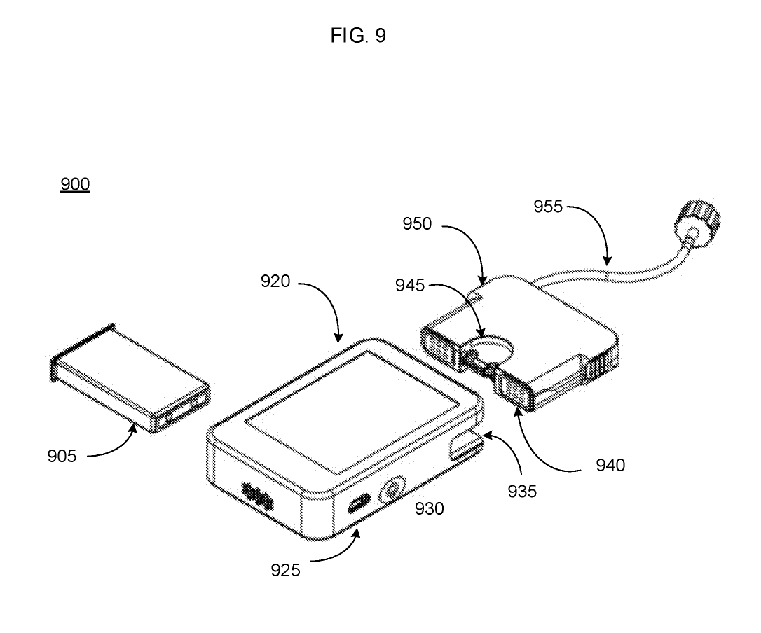

Referring now to FIG. 9, an exemplary drug delivery communication system 900 is illustrated, wherein the drug delivery communication system 900 may comprise a power source 905, a smart cartridge 950, and a master control unit 920. In some aspects, the power source 905 may be removable, which may allow for replacement of a drained power source 905, such as through a new power source 905 or through external recharging. In some embodiments, the power source 905 may be rechargeable through a micro USB port 925. In some implementations, the micro USB port 925 may allow for a transfer of data from the master control unit to an external device, such as a smartphone, memory stick, or computing device.

In some embodiments, the master control unit 920 may comprise a smart cartridge recess 935 configured to receive a smart cartridge 950. In some aspects, the smart cartridge 950 may comprise target connectors 940 that may initiate electrical communication with the master control unit 920 when the smart cartridge 950 is inserted into the smart cartridge recess 935 and the target connectors 940 engage with the master control unit 920. In some implementations, the target connectors 940 may allow for communication between the master control unit 920 and a pump actuation system 945, which may cause delivery of a medicament from the smart cartridge 950 through the outlet tubing 955.

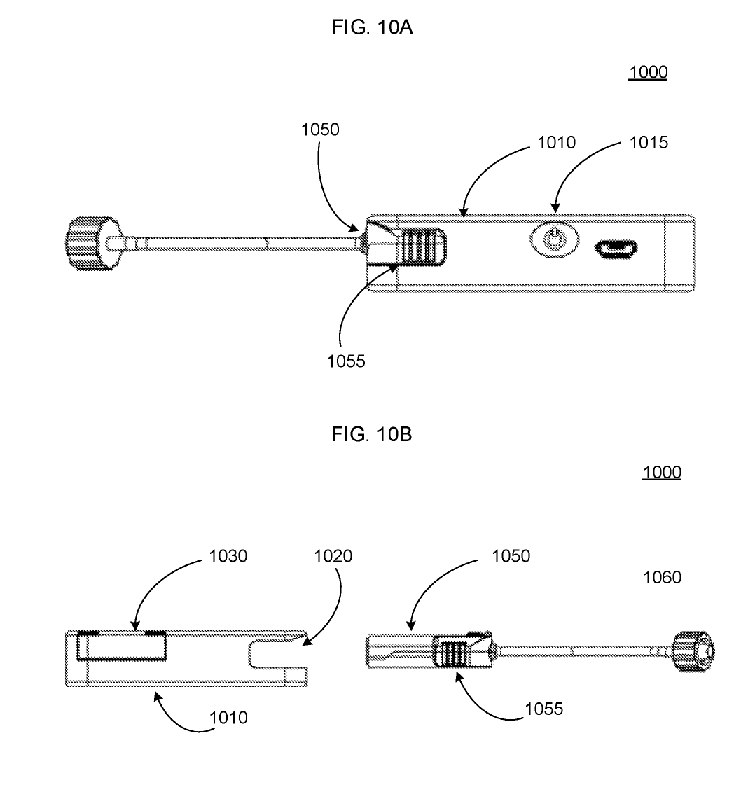

Referring now to FIG. 10A-10B, side views of an exemplary drug delivery communication system 1000 are illustrated, wherein the drug delivery communication system 1000 may comprise a power source 1030, a smart cartridge 1050, and a master control unit 1010. In some aspects, a smart cartridge 1050 may comprise a release mechanism 1055 that may be utilized to release or engage the smart cartridge 1050 into the smart cartridge recess 1020 in a master control unit 1010. In some embodiments, the power source 1030 may be fitted into a power source recess, which may engage electrical communication between the master control unit 1010 and the power source 1030. In some implementations, the master control unit 1010 may comprise a power switch 1015 that may allow a user to initiate or terminate power to and/or communication between one or more of the drug delivery communication system 1000, smart cartridge 1050, master control unit 1010, or other external device, such as a drug delivery base (not shown).

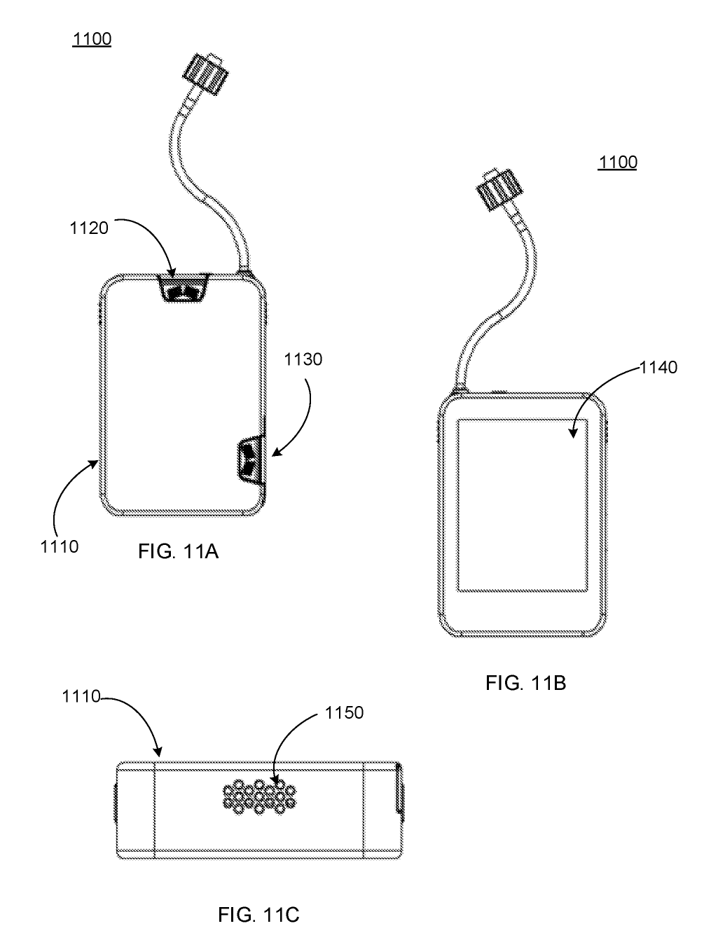

Referring now to FIGS. 11A-11C, different perspectives of a drug delivery communication system 1100 are illustrated. In some aspects, a smart cartridge 1120 may be inserted into a recess at one end of a master control unit 1110, and a power source 1130 may be inserted into a recess at one side of the master control unit 1110. In some aspects, the power source 1130 may be inserted during manufacturing, wherein the power source 1130 may not be removable after the manufacturing process, such as by a doctor, pharmacist, or user. In some aspects, a master control unit 1110 may comprise ventilation 1150 that may allow for a release of hot air, reducing risk of overheating.

In some implementations, a master control unit 1110 may comprise a user interface 1140 that may accept and receive control prompts from a user. The user interface 1140 may allow a user to program the drug delivery communication system 1100. For example, a user may directly input delivery information, such as a dosage, time of administering, and duration. A user may input intended medicament triggers, such as consumption of beverages or food that may cause a spike or drop in glucose levels. In some embodiments, the user interface 1140 may allow a user to customize notifications and tracking to suit the user's preferences.

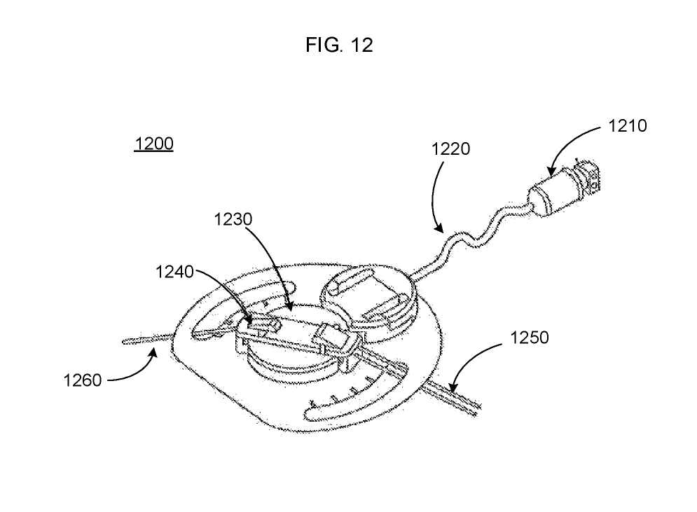

Referring now to FIG. 12, a perspective view of an exemplary drug delivery base 1200 is illustrated, in accordance with an embodiment of the present disclosure. In some aspects, the drug delivery base 1200 may be configured to deliver multiple medicaments. In some embodiments, the drug delivery base 1200 may comprise a continuous glucose monitoring system that may be integrated through a sensor cannula 1260. In some implementations, the drug delivery base 1200 may comprise a pump outlet 1210, a connector 1220, a base platform 1230, a rotating platform 1240, medicament delivery cannulae 1250, and a sensor cannula 1260.

In some aspects, a connector 1220 may comprise a plurality of connection regions. In some embodiments, a connector 1220 may comprise a region that may connect to a pump outlet 1210. In some aspects, a connector 1220 may comprise a region that may connector to the base platform 1230. In some implementations, medicament delivery cannulae 1250 may be configured to deliver one or more medicaments, such as from a smart cartridge through a master control unit as illustrated in FIG. 9. In some aspects, a sensor cannula 1260 may allow for the monitoring of one or more parameters, such as glucose levels.

In some embodiments, cannulae 1240, 1250 may be configured to penetrate a user's skin for placement within a user's blood stream for extended periods of time, such as days. Cannulae 1240, 1250 may comprise one or more materials, which may be flexible, rigid, or both, wherein a coating of the material may add functionality, such as sterility, limiting chance of breaking or bending, limit permeability of the material. For example, one or more of the cannulae 1240, 1250 may be coated with anticoagulation, hypoallergenic, anti-inflammatory, antibiotic agents, or combinations, thereof.

In some aspects, the number of cannulae 1240, 1250 may be increased or decreased depending on the needs of the user. For example, a user may require a plurality of medical delivery cannulae 1250 to receive a plurality of medicaments, and monitoring of a range of parameters may require a plurality of sensor cannulae 1240. Sensor cannulae 1240 may monitor attributes related to the delivered medicament, unrelated, or both. For example, where the delivered medicament comprises insulin, a related attribute may comprise glucose monitoring, and an unrelated parameter may comprise monitoring levels of a medicament taken for a separate disorder, such as epilepsy.

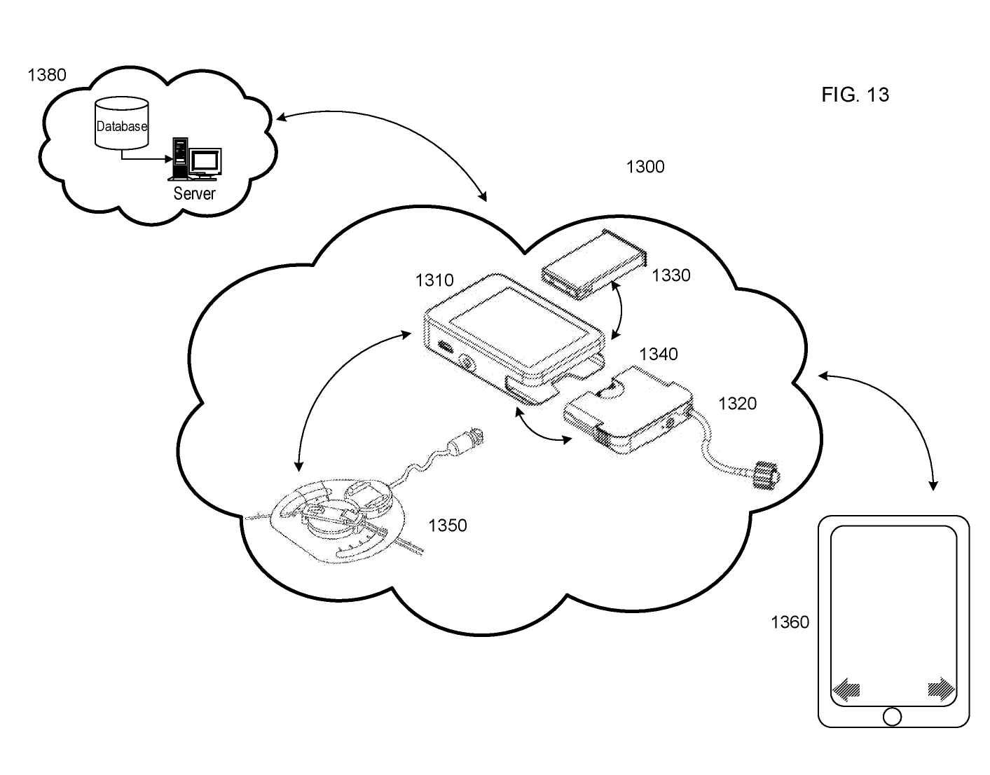

Referring now to FIG. 13, communication exchange within a drug delivery communication system 1300 and with external devices 1360, 1380 is illustrated. In some aspects, communication may occur between components of a drug delivery communication system 1300, such as between a master control unit 1310, smart cartridge 1320, power source 1330, pump actuation system 1340, and drug delivery base 1350. In some embodiments, direct communication may be practical, such as where there is electrical contact between the components. For example, direct communication between the smart cartridge 1320, pump actuation system 1340, and master control unit 1310 may be practical as the smart cartridge 1320 is inserted into the master control unit 1310, wherein power and control occurs through contact at target connectors, such as illustrated in FIG. 1. The pump actuation system 1340 may be indirectly in contact with the master control unit 1310 through the smart cartridge 1320 or directly.

In some aspects, the power source 1330 may be integrated within the master control unit 1310, wherein the power source 1330 may be rechargeable, such as through a charging port. In some embodiments, the power source 1330 may be removable, wherein the power source 1330 may comprise an independent communication mechanism, wherein the power source 1330 may communicate with the master control unit 1310 when inserted into a power docking receiver. In some aspects, the master control unit 1310 may provide adjustable and variable power to the smart cartridge 1320, wherein the power level may be based on data received from one or both the smart cartridge 1320 and drug delivery base 1350, such as from a micropump body, microprocessor, pump sensors, catheter with multiple channels, glucose sensor, or other communication and monitoring mechanism contained within one or more the smart cartridge 1320, master control unit 1310, and drug delivery base 1350.

In some implementations, wireless communication may be practical where electrical contact may not occur between communication components. For example, connecting the outlet tube of a smart cartridge 1320 to a drug delivery base connector may not establish direct electrical communication between the drug delivery base 1350 and one or more of the smart cartridge 1320, master control unit 1310, or pump actuation system 1340. In some aspects, sensors may monitor and detect when components are one or both mechanically and electrically connected, wherein detection may initiate or terminate communication between components.

In some aspects, a smart cartridge 1320 may contain a microprocessor that may perform flow sensor data acquisition, analysis, and communication with the master control unit 1310. In some embodiments, the communication between the smart cartridge 1320 and the master control unit 1310 may correct and adjust in real time the desired drug delivery. In some implementations, the smart cartridge 1320 may comprise one or more temperature sensor, vibration sensor, or other monitoring elements, which may provide a safety mechanism. For example, where a temperature may exceed a safe threshold temperature for a drug, the smart cartridge 1320 may communicate the temperature data to the master control unit 1310, and the master control unit 1310 may shut down access to the compromised compressible reservoir.

In some embodiments, a drug delivery communication system 1300 may communicate with external devices, such as an external server 1380 and external portable device 1360. In some aspects, an external server 1380 may be cloud storage for a user, medical provider, manufacturer, or other potentially interested entity. In some implementations, an external server 1380 may store and/or process data received from a drug delivery communication system 1300, such as communication data, delivery data, or status data.

As an example, communication data may comprise data related to when components are communicating; communication trigger events, such as emergency situations, repair notifications, or component replacement; or communication content, such as delivery prompts or replacement prompts. Delivery data may include data related to dosage, medicaments, duration of dosage, time between dosage, delivery trigger events, type of delivery (standard or emergency), or rate of delivery, as non-limiting examples. Status data may include data related to remaining medicament, power source levels, replacement events, tube effectiveness (no blockage or kinks), contamination, or glucose levels of a user, as non-limiting examples.

In some embodiments, a drug delivery communication system 1300 may communicate with an external portable device 1360, such as a smartphone. In some aspects, the external portable device 1360 may allow a user to monitor data from the drug delivery communication system 1300, such as communication data, delivery data, and status data. In some implementations, an external portable device 1360 may be integrated into the drug delivery communication system 1300, wherein the external portable device 1360 may have some control over one or more of the components.

In some aspects, a master control unit 1310 may be configured to receive data from external devices 1360, 1380, such as a smartphone, administering device, smart cartridge, or other communication device. In some aspects, the data may transmitted and received wirelessly, such as through Bluetooth, RFID, Near Field Communications (NFC), or other wireless network. In some embodiments, the data may be transmitted and received through a direct connection with the external devices 1360, 1380. In some implementations, sensors may be integrated in one or more communication systems, wherein sensor data may be shared over the communication systems.

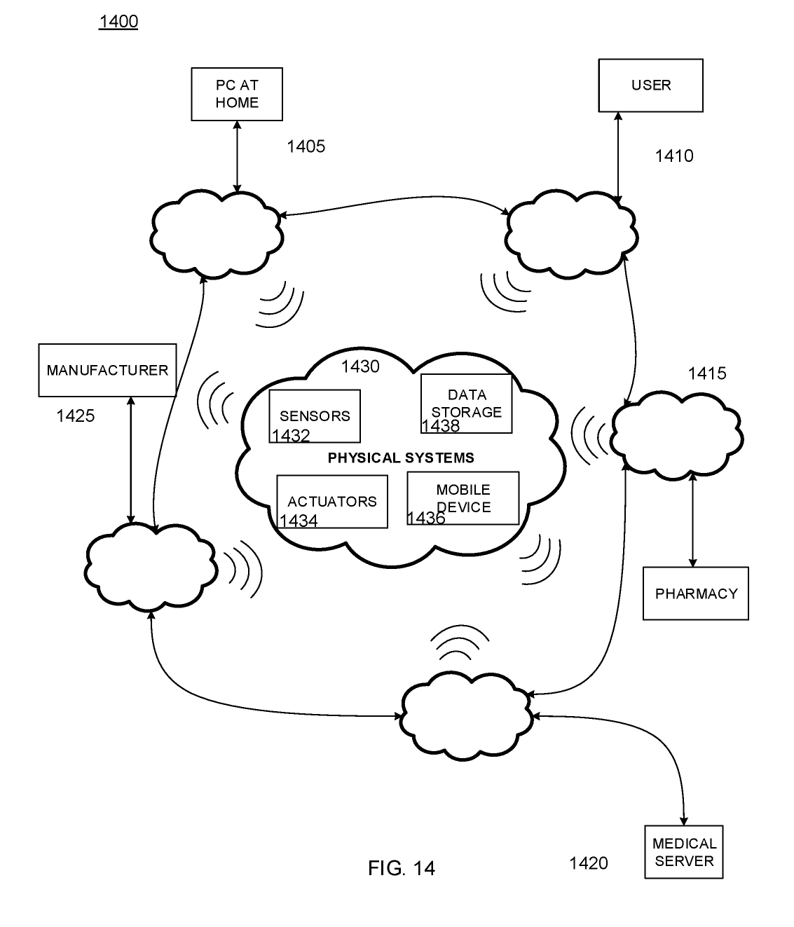

Referring now to FIG. 14, an exemplary cyber physical healthcare system 1400 is illustrated. In some aspects, data may be collected and stored by one or more independent servers, such as by a home computer 1405, user 1410, pharmacy 1415, medical provider 1420, or clinician 1425, as non-limiting examples. The collected and stored data may exchange data with physical systems, such as sensors 1432, actuators 1434, mobile devices 1436, and personal data storage 1438, as non-limiting examples.

In some aspects, personal data storage 1438 may be located on a wearable or portable device that may collect data directly from the sensor mechanisms on components of a drug delivery communication system or through a primary communication device, such as the master control unit. In some embodiments, the data exchanged between the physical systems and cyber systems may utilize one or more wireless communication systems and wired systems. In some implementations, data may be exchanged between cyber systems, such as between a medical provider 1420 and pharmacy 1415. For example, a pharmacy 1415 may transmit refill dates of the medicament, which may provide some insight into the use patterns of the user.

As an illustrative example, a home computer 1405 and user 1410 may collect daily information from at least a portion of the physical systems 1430, such as the components of a drug delivery communication system. The collected data may be transferred to a manufacturer 1425, pharmacy 1415, or medical provider 1420 periodically, such as after a device malfunction, in anticipation of a refill, or prior to scheduled visits, respectively. Similarly, the manufacturer 1425, pharmacy 1415, or medical provider 1420 may exchange data about a user as necessary.

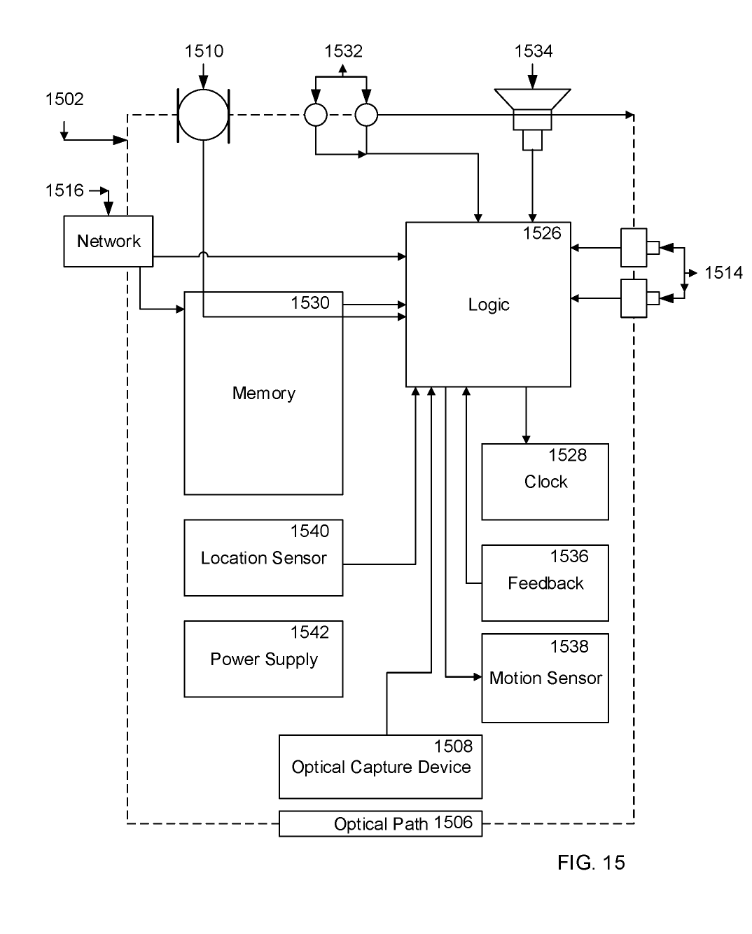

Referring now to FIG. 15, an exemplary block diagram of an exemplary embodiment of a mobile device 1502 is illustrated. The mobile device 1502 may comprise an optical capture device 1508, which may capture an image and convert it to machine-compatible data, and an optical path 1506, typically a lens, an aperture, or an image conduit to convey the image from the rendered document to the optical capture device 1508. The optical capture device 1508 may incorporate a Charge-Coupled Device (CCD), a Complementary Metal Oxide Semiconductor (CMOS) imaging device, or an optical sensor of another type.

In some embodiments, the mobile device 1502 may comprise a microphone 1510, wherein the microphone 1510 and associated circuitry may convert the sound of the environment, including spoken words, into machine-compatible signals. Input facilities 1514 may exist in the form of buttons, scroll-wheels, or other tactile sensors such as touch-pads. In some embodiments, input facilities 1514 may include a touchscreen display. Visual feedback 1532 to the user may occur through a visual display, touchscreen display, or indicator lights. Audible feedback 1534 may be transmitted through a loudspeaker or other audio transducer. Tactile feedback may be provided through a vibration module 1536.

In some aspects, the mobile device 1502 may comprise a motion sensor 1538, wherein the motion sensor 1538 and associated circuitry may convert the motion of the mobile device 1502 into machine-compatible signals. For example, the motion sensor 1538 may comprise an accelerometer, which may be used to sense measurable physical acceleration, orientation, vibration, and other movements. In some embodiments, the motion sensor 1538 may comprise a gyroscope or other device to sense different motions.

In some implementations, the mobile device 1502 may comprise a location sensor 1540, wherein the location sensor 1540 and associated circuitry may be used to determine the location of the device. The location sensor 1540 may detect Global Position System (GPS) radio signals from satellites or may also use assisted GPS where the mobile device may use a cellular network to decrease the time necessary to determine location. In some embodiments, the location sensor 1540 may use radio waves to determine the distance from known radio sources such as cellular towers to determine the location of the mobile device 1502. In some embodiments these radio signals may be used in addition to and/or in conjunction with GPS.

In some aspects, the mobile device 1502 may comprise a logic module 1526, which may place the components of the mobile device 1502 into electrical and logical communication. The electrical and logical communication may allow the components to interact. Accordingly, in some embodiments, the received signals from the components may be processed into different formats and/or interpretations to allow for the logical communication. The logic module 1526 may be operable to read and write data and program instructions stored in associated storage 1530, such as RAM, ROM, flash, or other suitable memory. In some aspects, the logic module 1526 may read a time signal from the clock unit 1528. In some embodiments, the mobile device 1502 may comprise an on-board power supply 1542. In some embodiments, the mobile device 1502 may be powered from a tethered connection to another device, such as a Universal Serial Bus (USB) connection.

In some implementations, the mobile device 1502 may comprise a network interface 1516, which may allow the mobile device 1502 to communicate and/or receive data to a network and/or an associated computing device. The network interface 1516 may provide two-way data communication. For example, the network interface 1516 may operate according to an internet protocol. As another example, the network interface 1516 may comprise a local area network (LAN) card, which may allow a data communication connection to a compatible LAN. As another example, the network interface 1516 may comprise a cellular antenna and associated circuitry, which may allow the mobile device to communicate over standard wireless data communication networks. In some implementations, the network interface 1516 may comprise a Universal Serial Bus (USB) to supply power or transmit data. In some embodiments, other wireless links known to those skilled in the art may also be implemented.

### CONCLUSION

A number of embodiments of the present disclosure have been described. While this specification contains many specific implementation details, these should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. A drug delivery communication system for use in conjunction with a smart cartridge system, the communication system comprising:
a master control unit device comprising:
a first electronic receiving body,
a power source,
a master control processor, wherein the master control processor is configured to control the power source;
a first smart cartridge device connectable to the master control unit, wherein communication initiates when the first smart cartridge is inserted into the first electronic receiving body, the first smart cartridge device comprising:
a first compressible reservoir configured to contain a first medicament;
a first internal tubing connected to the first compressible reservoir;
a pumping mechanism operably connected to the first internal tubing and configured to be electrically connected to the power source, wherein the pumping mechanism controls flow of the first medicament from the first compressible reservoir;
a flow rate sensor configured to monitor a flow of the first medicament from the first internal tubing to the first outlet tube;
a first outlet tube connected to the first internal tubing through which a first expulsion of the first medicament to an external body flows; and
a housing containing the first compressible reservoir, the first internal tubing, the pumping mechanism, the flow rate sensor, and at least part of the first outlet tube; and
a first smart cartridge processor configured to receive a first set of delivery data from the master control processor and transmit a second set of delivery data to the master control processor, wherein the first set and the second set of delivery data at least partially controls the delivery of the first medicament to a user and the delivery of power to the first smart cartridge.

2. The drug delivery communication system of claim 1, wherein at least a portion of communication is wireless.

3. The drug delivery communication system of claim 1, wherein the second set of delivery data comprise data from one or more the first compressible reservoir, the first internal tubing, the pumping mechanism, the flow rate sensor, the first outlet tube, the housing, and the first smart cartridge processor.

4. The drug delivery communication system of claim 1, wherein the pumping mechanism comprises a pump actuation system with a pump processor configured to receive pump data from one or both the master control processor and the first cartridge processor, wherein the pump data controls an actuation of the pump actuation system.

5. The drug delivery communication system of claim 4, wherein the second set of delivery data comprises delivery data from one or both the pump actuation system and pump processor.

6. The drug delivery communication system of claim 3, wherein the master control unit is configured to receive a first set of status data from the first smart cartridge device, wherein the first set of status data relates to a status of one or more the master control unit, the first compressible reservoir, the first internal tubing, the pumping mechanism, the flow rate sensor, the first outlet tube, the housing, and the first smart cartridge processor.

7. The drug delivery communication system of claim 6, further comprising a drug delivery base in communication with one or both the smart cartridge and the master control unit, the drug delivery base comprising:
a first drug delivery base processor configured to transmit a third set of delivery data from the master control processor, wherein the delivery data at least partially controls the delivery of the first medicament to a user;
a first receiving channel connectable to the first outlet tube;
a first dispensing channel configured to deliver medicament subcutaneously;
a platform configured to secure to a skin surface of a user, wherein the platform secures the first outlet tube to the first dispensing channel and stabilizes the first dispensing channel to the user.

8. The drug delivery communication system of claim 7, wherein the status data further relates to a status of one or more of the first drug delivery base processor, the first receiving channel, the first dispensing channel, and the platform, and wherein the second set of delivery data comprises delivery data from one or more of the first drug delivery base processor, the first receiving channel, the first dispensing channel, and the platform.

9. The drug delivery communication system of claim 6, wherein the first compressible reservoir comprises:
a first fill port configured to accept a filling mechanism, wherein the filling mechanism is configured to add a first medicament to the first compressible reservoir;
a first overflow port configured to dispel an excess amount of the first medicament where the first medicament filled exceeds a first threshold volume capacity within the first compressible reservoir;
a first flow port through which the first medicament flows for use; and
a first flexible pouch configured to contain the first medicament.

10. The drug delivery communication system of claim 9, wherein the status data further relates to a status of one or more of the first fill port, first overflow port, first flow port, and the first flexible pouch, and wherein the second set of delivery data comprises delivery data from one or more of the first fill port, first overflow port, first flow port, and the first flexible pouch.

11. The drug delivery communication system of claim 1, wherein the master control unit device further comprises an interface configured to receive delivery input from the user, wherein the delivery data is based at least in part on the delivery input.

12. The drug delivery communication system of claim 8, further comprising a first external communication device configured to receive one or more the first set of status data, the first set of delivery data, the second set of delivery data, and the third set of delivery data.

13. A drug delivery communication system for use in conjunction with a smart cartridge system, the communication system comprising:
a master control unit device comprising:
a first electronic receiving body,
a power source,
a master control processor, wherein the master control processor is configured to control the power source;
a smart cartridge device connectable to the master control unit, wherein the smart cartridge comprises:
a first compressible reservoir configured to contain a first medicament;
a first internal tubing connected to the first compressible reservoir;
a second compressible reservoir configured to contain a second medicament;
a second internal tubing connected to the second compressible reservoir;
a pumping mechanism operably connected to the first internal tubing and the second internal tubing and configured to be electrically connected to the power source, wherein the pumping mechanism controls flow of the first medicament from the first compressible reservoir and flow of the second medicament from the second compressible reservoir;
a first outlet tube connected to the first internal tubing through which a first expulsion of the first medicament to an external body flows;
a second outlet tube connected to the second internal tubing through which a first expulsion of the second medicament to the external body flows; and
a flow rate sensor configured to monitor a flow of the first medicament from the first internal tubing to the first outlet tube and a flow of the second medicament from the second internal tubing to the second outlet tube;
a housing containing the first compressible reservoir, the first internal tubing, the pumping mechanism, the flow rate sensor, and at least part of the first outlet tube;
a first smart cartridge processor configured to receive a first set of delivery data from the master control processor and transmit a second set of delivery data to the master control processor, wherein the first set and the second set of delivery data at least partially controls the delivery of the first medicament and the second medicament to a user and the delivery of power to the first smart cartridge.

14. The drug delivery communication system of claim 13, wherein the second set of delivery data comprise data from one or more the first compressible reservoir, the second compressible reservoir, the first internal tubing, the second internal tubing, the pumping mechanism, the flow rate sensor, the first outlet tube, the second outlet tube, and the housing, the first smart cartridge processor.

15. The drug delivery communication system of claim 13, wherein the pumping mechanism comprises a pump actuation system with a pump processor configured to receive pump data from one or both the master control processor and the first cartridge processor, wherein the pump data controls an actuation of the pump actuation system.

16. The drug delivery communication system of claim 14, wherein the master control unit is configured to receive a first set of status data from the first smart cartridge device, wherein the first set of status data relates to a status of one or more the master control unit, the first compressible reservoir, the second compressible reservoir, the first internal tubing, the second internal tubing, the pumping mechanism, the flow rate sensor, the first outlet tube, the second outlet tube, and the housing, the first smart cartridge processor.

17. The drug delivery communication system of claim 16, further comprising a drug delivery base in communication with one or both the smart cartridge and the master control unit, the drug delivery base comprising:
a first drug delivery base processor configured to transmit a third set of delivery data from the master control processor, wherein the delivery data at least partially controls the delivery of the first medicament to a user;
a first receiving channel connectable to the first outlet tube;
a first dispensing channel configured to deliver the first medicament subcutaneously;
a second receiving channel connectable to the second outlet tube;
a second dispensing channel configured to deliver the second medicament subcutaneously;
a platform configured to secure to a skin surface of a user, wherein the platform secures the first outlet tube to the first dispensing channel, the second outlet tube to the second dispensing channel and stabilizes the first dispensing channel and the second dispensing channel to the user.

18. The drug delivery communication system of claim 17, wherein the status data further relates to a status of one or more of the first drug delivery base processor, the first receiving channel, the first dispensing channel, and the platform, and wherein the second set of delivery data comprises delivery data from one or more of the first drug delivery base processor, the first receiving channel, the first dispensing channel, and the platform.

19. The drug delivery communication system of claim 13, wherein the master control unit device further comprises an interface configured to receive delivery input from the user, wherein the delivery data is based at least in part on the delivery input.

20. The drug delivery communication system of claim 18, further comprising a first external communication device configured to receive one or more the first set of status data, the first set of delivery data, the second set of delivery data, and the third set of delivery data.
